(19) United States
(12) Patent Application Publication
(10) Pub. No.: US 2025/0377358 A1
(43) Pub. Date: Dec. 11, 2025

(54) NOVEL BIOMARKER TO PREDICT EFFICACY OF CANCER IMMUNOTHERAPY

(71) Applicant: Hoffmann-La Roche Inc., Little Falls, NJ (US)

(72) Inventors: Jehad Charo, Wettswil (CH); David Dejardin, Delémont (CH)

(73) Assignee: Hoffmann-La Roche Inc., Little Falls, NJ (US)

(21) Appl. No.: 19/205,721

(22) Filed: May 12, 2025

Related U.S. Application Data
(63) Continuation of application No. PCT/EP2023/081548, filed on Nov. 13, 2023.

(30) Foreign Application Priority Data
Nov. 14, 2022 (EP) 22207100.3

(51) Int. Cl. G01N 33/569; G01N 33/574

(52) U.S. Cl. CPC G01N 33/56972; G01N 33/57484

(57) ABSTRACT

The present invention provides a novel biomarker comprising a composite score of T cell density in paired biopsies taken before and after treatment of a patient, with cancer, to inform potential outcomes in immunotherapy, including but not limited to clinical trials. The invention also provides methods to determine whether a cancer patient is likely to benefit from immunotherapy by using said biomarker.

### CROSS-REFERENCE TO RELATED APPLICATIONS

This application is continuation of the PCT Application No. PCT/EP2023/081548, filed Nov. 13, 2023, which claims the benefit of European Application No. 22207100.3, filed Nov. 14, 2022, each of which is incorporated by reference in its entirety.

### FIELD OF THE INVENTION

The present invention relates to novel biomarkers for determining whether a patient, with cancer, is likely to benefit from treatment with immunotherapy.

### BACKGROUND OF THE INVENTION

Immunotherapy of cancer that involves harnessing the patients' own immune system for anticancer effects has rapidly advanced over the last two decades. One of the earliest effective immunotherapies against melanoma and renal cell cancer was interleukin 2 (IL-2) [1]. More recently, the Nobel prize-winning discovery of checkpoint molecules such as CTLA-4 and PD-1/ PD-L1 led to the development of a new treatment approach called cancer immunotherapy that has transformed the landscape of cancer therapy [2]. These “checkpoint inhibitors” or CPI, drugs that inhibit checkpoint molecules have demonstrated unprecedented clinical efficacy against a variety of different cancer types [3]. Presently there are a numerous immunotherapy drug and combination candidates that are being tested in clinical trials against diverse tumor types.

The clinical testing of immunotherapy drugs brings unique challenges that are different from conventional cytotoxics. The response patterns, kinetics, and mechanism of action of immunotherapy differ fundamentally from cytotoxics or tyrosine kinase inhibitors. Additionally, there are few pharmacodynamic biomarkers for immunotherapy drug candidates that can effectively measure biological effects of an administered immunotherapy drug on patients and inform on the clinical benefit from such a treatment [4]. There is therefore the unmet need for markers that can provide early information on the potential outcome of clinical trials, in addition to and before the conventional endpoints such as overall response rates, progression free survival and overall survival are reached at the end of the study period.

The work of Jerome Galon and others have underscored the significance of T cell infiltrate in the tumor milieu, particularly CD8+ T cells and its relationship to clinical prognosis [5]. There is now a worldwide consensus that conventional classification of patients based on AJCC/TNM systems provide limited prognostic information and that incorporation of an immune-based classification or “immunoscore” is an essential diagnostic and prognostic tool for clinical decision making [6].

The present inventors have explored the density of CD8+ T cells as a putative, early marker of clinical, or therapeutic, efficacy. Previously, Petrelli F et al [7] have described three criteria for an ideal biomarker: 1) direct association between the disease mechanism, the biomarker, and the clinical endpoint; 2) change in biomarker should be associated with change in disease status for individual patients, 3) association between a change in biomarker caused by a therapeutic intervention and the ultimate clinical outcome within a trial. Using these criteria, the present inventors have examined changes in CD8+T cell density in paired tumor biopsies taken before and after treatment, across several different immunotherapy, early-phase, trials. They demonstrated that a composite decision rule based on CD8+T cell density correlates with clinical outcome both at the level of individual patients as well as collectively for the study. Furthermore, the present invention provides specific cut-off threshold values that can be used to predict potential outcomes of any immunotherapy treatment, including but not limited to early clinical trials, where the mechanism of action involves expansion of CD8 T cells to mediate antitumor response.

The present invention thus provides a framework for decision making whether or not to proceed with cancer immunotherapy in a patient or in a clinical study, for example in early cancer immunotherapy clinical studies, based on the use of selected biomarker data. As a prototype, we evaluated whether these decisions can be made utilizing on-treatment CD8 T cell density in tumor microenvironment.

### BRIEF DESCRIPTION OF THE FIGURES

FIGS. 1A and 1B: Forest plot of HR identifies total CD8 T cell density as the most consistent response biomarker in simlukafusp alfa studies; FIG. 1A with Baseline (BL) correction, without BL correction.

FIGS. 2A and 2B: Higher total CD8 T cell density is associated with better response in simlukafusp alfa studies. Distribution of

FIG. 2A log FC and

FIG. 2B log OTD for each response category.

FIGS. 3A, 3B, 3C and 3D: Derivation of thresholds separating higher and lower risk of progression:

FIG. 3A The true positive fraction and false positive fraction given by the ROC curve for FC shows an optimal threshold of 1.3 log FC

FIG. 3B The true positive fraction and false positive fraction given by the ROC curve for OTD shows an optimal threshold of 6.7 log OTD

FIG. 3C Repeated landmark analysis on PFS shows lowest optimal threshold for log FC as 0.9

FIG. 3D Repeated landmark analysis on PFS shows lowest optimal threshold for log OTD as 6.2

FIGS. 4A and 4B: Prolonged PFS is associated with total CD8 T cell density as a response biomarker in training and validation data sets with a Log FC 0.9 fold change threshold FIG. 4A and 6.2 log OTD value threshold FIG. 4B.

FIG. 5: Distribution of effect of being above threshold on growth and shrinkage of tumor.

FIG. 6: Low correlation between log FC and OTD of total CD8 T cell density underscores the potential for an approach combining both readouts.

FIG. 7: Readout of data from an experiment with n=15.

FIG. 8: Probability of success of the decision rule given true OTD and FC for studies of 5, 10, 20, 60 patients with paired biopsy data.

### SUMMARY OF THE INVENTION

In one embodiment, the present invention provides a method to determine whether a patient, with cancer, is likely to benefit from treatment with immunotherapy comprising
a) obtaining samples from that patient before (at baseline) and after treatment,
b) determining the fold increase (FC) in CD8+T-cells and/or the on-treatment density (OTD) of CD8+T-cells in the sample after treatment compared to the sample at baseline,
c) comparing values obtained in b) to threshold values, wherein
the patient is likely to benefit from said treatment if the values obtained in b) are above the threshold.

In another embodiment, the present invention provides a biomarker for use in the above-mentioned method.

In another embodiment, the present invention provides a method of treating patients, with cancer, using the biomarker as defined herein.

In still another embodiment, the present invention provides the biomarker as defined herein for use in monitoring treatment of a patient, with cancer.

In still another embodiment, the present invention provides the biomarker as defined herein for use to enable the decision whether a patient, with cancer, continues treatment.

In still another embodiment, the present invention provides the biomarker as defined herein for use to enable the decision whether to continue or to stop a clinical study.

### DETAILED DESCRIPTION OF THE INVENTION

The association of T cells, specifically CD8+T cells in the tumor, and better clinical outcome is

not a new concept, including in cancer immunotherapy (16). Bocchialini et al. [17] reported high densities of CD8+ tumor infiltrating lymphocytes (TIL) correlated with improved freedom from recurrence and cause-specific survival in patients with thymic carcinomas. Similar results have been reported for CD8+ TIL and improved overall survival and recurrence-free survival in oral squamous cell carcinoma [18, 19]. The report by Galon et al. demonstrated that immune cell infiltrate in colorectal cancers correlate to a better extent with clinical outcome than conventional histological staging [20].

The present inventors have analyzed CD8 T-cell density in paired biopsies across 8 phase I and phase II studies, investigating the dose safety and clinical benefit of 5 investigational drugs, as single agent or in combination with atezolizumab, cetuximab or bevacizumab. The novel aspects of their work are essentially as follows: A) Using the data from the paired biopsies of the FAP-IL2v studies as the training set, it is demonstrated that fold-change (FC), and on-treatment change in density of CD8+ T cells (OTD) correlate with clinical outcome. B) Validating the utility of FC and OTD as a biomarker for predicting clinical outcome across a variety of different immunotherapy, early-phase trials that tested Immuno-Oncology (I-O) candidates independently, or in combination with other drugs. C) Identifying thresholds for FC and OTD which could be used to dissociate potential responders from non-responders. D) Presenting a method for using FC or OTD in combination to inform early on, the clinical outcomes both for an early-phase clinical trial, as well as at the level of an individual patient. Among other things, this approach reduces the impact of timing the sampling point for on-treatment biopsy on determining outcome of a drug treatment.

Therefore, the present invention solves the problem of providing an effective biomarker that can provide early information on outcomes and responses, for example in investigational (early) clinical trials, but also in established cancer therapy using immunotherapy. In a clinical trial setting, it facilitates conservation of resources and efforts that would otherwise be expended to complete a clinical trial which has low probability of success. More importantly, in a clinical trial setting as well as in therapy, it reduces the risk of harm to patients by limiting exposure to ineffective therapies and expedites switching to potentially more effective treatment modalities.

The present invention demonstrates strong association between high levels of CD8 on-treatment (OTD) and clinical benefit of treatment and between increased level of CD8 on-treatment vs baseline (FC) and clinical benefit of treatment. While either OTD or FC can qualitatively be sufficient for informing about likelihood of clinical outcome, a set threshold for each of them is required for concluding on this information. The composite biomarker in accordance with the present invention builds on capturing the dynamic change over a set study period by factoring these two values. An on-treatment expansion of CD8+ cells which results in a positive FC, also has the consequence of increase in OTD as a result of accumulation of CD8+ cells in the tumor milieu. Using both these values mitigates the need for precisely timing the on-treatment biopsy at a moment when the change is maximal. Instead, the OTD reflects a median change in CD8+ infiltration over a period of time. Equally important is the aspect that depending on the mechanism of action of an immunotherapy drug, most of which rely on the pre-existing immunity against the tumor, a minimal threshold for either FC or OTD is needed to inform on potential clinical-, or therapeutic, outcome. As a corollary, a change in either of the two parameters can inform on the potential outcome with the greatest confidence in prediction being obtained when both parameters are considered in tandem.

Therefore, in one aspect the present invention provides a new method for using changes in CD8+ cells as a surrogate marker for providing early information on the clinical outcome for early-phase I-O trials and describes an algorithm which can be used to generalize the application of this method across different I-O drug trials in diverse cancer types. In another aspect the present invention also provides that same method as a biomarker for obtaining early information whether a patient, with cancer, benefits from treatment with immunotherapies,

Therefore, in one aspect, the present invention provides a method to determine whether a patient, with cancer, is likely to benefit from treatment with immunotherapy.

The terms “cancer”, “tumor”, “tumour”, and “carcinoma”, are used interchangeably herein to refer to cells that exhibit relatively abnormal, uncontrolled, and/or autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. The teachings of the present disclosure may be relevant to any and all cancers. To give but a few, non-limiting examples, in some embodiments, teachings of the present disclosure are applied to one or more cancers such as, for example, hematopoietic cancers including leukemias, lymphomas (Hodgkins and non-Hodgkins), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like. In one aspect, a cancer in accordance with the present invention is a solid tumor. In another aspect, a cancer in accordance with the present invention is a hematological tumor. In yet another aspect the cancer is a solid tumor selected from lung cancer (including non-small cell lung cancer), breast cancer, thyroid cancer, head and neck cancer, pancreatic cancer, prostate cancer, bladder cancer, colon cancer, esophageal cancer, ovarian cancer, gastric cancer, skin cancer and colorectal cancer.

The terms “immunotherapy” or “cancer immune therapy” or “cancer immunotherapy” can be used interchangeably and are known to a person of skill in the art, for example, a clinical oncologist. In one aspect, “cancer immunotherapy” means a therapeutic treatment that stimulates or restores the ability of the immune system to fight cancer by inducing, enhancing or suppressing an immune response. Cancer immunotherapy results in targeted immune activity against a disease-specific antigen, either by increasing immune cell recognition of the target or by reducing disease-related immune suppression. In one aspect of the present invention “cancer immune therapy” means any therapy where the mechanism of action involves expansion of CD8 T cells to mediate antitumor response. In another aspect the immunotherapy in accordance with the present invention is selected from Immune Checkpoint Inhibitors; T-cell transfer therapy (CAR T-cells); monoclonal, mono-or multispecific, preferably bispecific, antibodies; cancer vaccines; or other immune system modulators such as, for example, cytokines. In one aspect the immunotherapy is an approved therapy or an investigational therapy. In another aspect of the present invention “cancer immune therapy” means the “investigational drugs” disclosed in the accompanying working examples.

The immunotherapy in accordance with the present invention may also include combination therapy. As used herein, the term “combination therapy” refers to those situations in which a subject is exposed to two or more therapeutic regimens (e.g., two or more therapeutic agents). In some embodiments, two or more agents may be administered simultaneously, either in a combined-or separated dosage forms. Alternatively, such agents may be administered sequentially which may also include overlapping dosing regimens. In some aspects the combination partner is another immunotherapeutic drug. In one aspect the drug for use in the immunotherapy in accordance with the present invention (the “immunotherapeutic drug”) is used in combination with atezolizumab, trastuzumab, cetuximab or bevacizumab. In one aspect atezolizumab, trastuzumab, cetuximab or bevacizumab are administered separately according to their approved doses, dosage regimen and dosage forms.

The term “patient” as used herein means one or several individuals suffering from cancer. In one aspect, the term “patient” means an individual suffering from cancer. In another aspect, the term “patient” means a group of individuals such as, for example, as study group in clinical trials. In that aspect, the composite decision rule in accordance with the present invention can be used to decide whether a given clinical trial involving e.g. an immunotherapy should be continued, the regimen modified or even stopped.

The term “benefit from treatment” as used herein refers to clinical intervention in an attempt to alter the natural course of a disease in the individual being treated, and can be performed either for prophylaxis or during the course of clinical pathology. Such benefit includes cellular or biological responses, a complete response, a partial response, a stable disease (without progression or relapse), or a response with a later relapse of the patient from or as a result of the treatment with the cancer immunotherapy drug. For example, an effective response can be reduced tumor size, progression-free survival, or overall survival. In one aspect of the invention a patient benefits from treatment if he/she shows improved progression-free-survival (PFS) or partial—or complete response. In that aspect, the patient benefits “in the form of” e.g. PFS, as sometimes also used herein.

A “sample” as used herein means a sample of a body fluid, a sample of separated cells or a sample from a tissue or an organ. Samples of body fluids can be obtained by well-known techniques and include, samples of blood, plasma, serum, urine, lymphatic fluid, sputum, ascites, bronchial lavage or any other bodily secretion or derivative thereof. Tissue or organ samples may be obtained from any tissue or organ by, e.g., biopsy. Separated cells may be obtained from the body fluids or the tissues or organs by separating techniques such as centrifugation or cell sorting. E.g., cell-, tissue-or organ samples may be obtained from those cells, tissues or organs which express or produce the biomarker. The sample may be frozen, fresh, fixed (e.g. formalin fixed), centrifuged, and/or embedded (e.g. paraffin embedded), etc. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, biopsies may also be subjected to postcollection preparative and storage techniques, e.g., fixation. In one preferred embodiment, a “sample” is a tumor biopsy sample from the patient.

“Determining the fold increase (FC) in CD8+ T-cells and/or the on-treatment density (OTD) of CD8+ T-cells”, as used herein, can be carried out according to methods known to the person of skill in the art [10]. In one aspect, said determination can be carried out using ROC curve analysis [21] or repeated landmark analysis [22]. In another aspect, said determination can be carried out as described in the accompanying working examples.

In one embodiment, the present invention provides a method to determine whether a patient, with cancer, is likely to benefit from treatment with immunotherapy comprising
a) obtaining samples from that patient before (at baseline) and after treatment,
b) determining the fold increase (FC) in CD8+ T-cells and/or the on-treatment density (OTD) of CD8+ T-cells in the sample after treatment compared to the sample at baseline,
c) comparing values obtained in b) to threshold values, wherein
the patient is likely to benefit from said treatment if the values obtained in b) are above the threshold.

In one embodiment the above method is an in vitro method.

In yet another embodiment, the present invention provides any of the above methods, wherein the sample is a patient's tumor biopsy sample.

In yet another embodiment, the present invention provides any of the above methods, wherein the cancer is a solid tumor.

In yet another embodiment, the present invention provides any of the above methods wherein the cancer is selected from lung cancer (including non-small cell lung cancer), breast cancer, thyroid cancer, head and neck cancer, pancreatic cancer, prostate cancer, bladder cancer, colon cancer and colorectal cancer.

In yet another embodiment, the present invention provides any of the above methods, wherein the patient benefit from treatment is either of a partial response (PR), a complete response (CR) or a longer progression-free survival (PFS).

In yet another embodiment, the present invention provides any of the above methods, wherein the patient is likely to benefit in the form of PFS if the threshold value for the fold increase in CD8+ T-cells, compared to baseline, is ≥0.9 on a log scale as determined by repeated landmark analysis.

In yet another embodiment, the present invention provides any of the above methods, wherein the patient is likely to benefit in the form of PR, CR and/or PFS if the threshold value for the on-treatment density of CD8+ T-cells, compared to baseline, is ≥6.2 on a log scale (approximately 500 cells/mm²) as determined by repeated landmark analysis.

In yet another embodiment, the present invention provides any of the above methods, wherein the patient is likely to benefit from immunotherapy if
the threshold value for the fold increase in CD8+ T-cells, compared to baseline, is ≥0.9 on a log scale as determined by ROC curve analysis, and
the threshold value for the on-treatment density of CD8+ T-cells, compared to baseline, is ≥6.2 on a log scale (approximately 500 cells/mm²) as determined by repeated landmark analysis.

In yet another embodiment, the present invention provides any of the above methods, wherein the patient is likely to benefit in the form of PR or CR if the threshold value for the fold increase in CD8+ T-cells, compared to baseline, is ≥1.3 on a log scale and the threshold value for the on-treatment density of CD8+ T-cells, compared to baseline, is ≥6.7 on a log scale as determined by ROC curve analysis.

In yet another embodiment, the present invention provides any of the above methods, wherein the patient is likely to benefit in the form of PFS if the threshold value for the fold increase in CD8+ T-cells, compared to baseline, is ≥0.9 on a log scale and the threshold value for the on-treatment density of CD8+ T-cells, compared to baseline, is ≥6.2 on a log scale as determined by repeated landmark analysis.

In yet another embodiment, the threshold values as defined herein are obtained using assays as described in [10]. In a preferred embodiment the threshold values as defined herein are obtained with the CD8/Ki67 assay as described in or in the accompanying working examples. In yet another embodiment, the present invention provides any of the above methods for use in early stage clinical trials.

In yet another embodiment, the present invention provides any of the above methods for use in monitoring treatment of a patient, with cancer, wherein the treatment involves an immunotherapy.

In yet another embodiment, the present invention provides any of the above methods for use to enable the decision whether a patient, with cancer, continues treatment with immunotherapy.

In still another embodiment, the present invention provides the biomarker as defined herein for use to enable the decision whether to continue or to stop a clinical study.

In yet another embodiment, the present invention provides a biomarker for use in determining whether a patient, with cancer, is likely to benefit from treatment with immunotherapy, wherein the biomarker is characterized by
the fold increase (FC) in CD8+ T-cells; and/or
the on-treatment density (OTD) of CD8+ T-cells
   in a sample obtained from that patient after treatment, compared to a sample of that same patient at baseline.

In still another embodiment, the present invention provides the biomarker for use as described above, wherein the biomarker is characterized by
the fold increase (FC) in CD8+ T-cells; or
the on-treatment density (OTD) of CD8+ T-cells
   in a sample obtained from that patient after treatment, compared to a sample of that same patient at baseline.

In still another embodiment, the present invention provides the biomarker for use as described above, wherein the biomarker is characterized by
the fold increase (FC) in CD8+ T-cells; and
the on-treatment density (OTD) of CD8+ T-cells
   in a sample obtained from that patient after treatment, compared to a sample of that same patient at baseline.

In still another embodiment, the present invention provides the biomarker for use as described in any of the previous embodiments, wherein the fold increase (FC) in CD8+ T-cells and/or the on-treatment density (OTD) of CD8+ T-cells is above their corresponding value detected at baseline.

In still another embodiment, the present invention provides the biomarker for use as described in any of the previous embodiments, wherein the values of fold increase (FC) in CD8+ T-cells or the on-treatment density (OTD) of CD8+ T-cells are detected by ROC curve analysis or repeated landmark analysis. In a preferred embodiment the values of fold increase (FC) in CD8+ T-cells or the on-treatment density (OTD) of CD8+ T-cells are detected as described in the accompanying working examples.

In still another embodiment, the present invention provides the biomarker for use as described in any of the previous embodiments, wherein the patient benefit from treatment is either of a partial response (PR), a complete response (CR) or a longer progression-free survival (PFS).

In still another embodiment, the present invention provides the biomarker for use as described in any of the previous embodiments, wherein the patient is likely to benefit in the form of PFS if the threshold value for the fold increase in CD8+ T-cells, compared to baseline, is ≥0.9 on a log scale as determined by repeated landmark analysis.

In still another embodiment, the present invention provides the biomarker for use as described in any of the previous embodiments, wherein the patient is likely to benefit in the form of PR, CR and/or PFS if the threshold value for the on-treatment density of CD8+ T-cells, compared to baseline, is ≥6.2 on a log scale (approximately 500 cells/mm²) as determined by repeated landmark analysis.

In still another embodiment, the present invention provides the biomarker for use as described in any of the previous embodiments, wherein the patient is likely to benefit from immunotherapy if
the threshold value for the fold increase in CD8+ T-cells, compared to baseline, is ≥0.9 on a log scale as determined by ROC curve analysis, and
the threshold value for the on-treatment density of CD8+ T-cells, compared to baseline, is ≥6.2 on a log scale (approximately 500 cells/mm²) as determined by repeated landmark analysis.

In still another embodiment, the present invention provides the biomarker for use as described in any of the previous embodiments, wherein the patient is likely to benefit in the form of PR or CR if the threshold value for the fold increase in CD8+ T-cells, compared to baseline, is ≥1.3 on a log scale and the threshold value for the on-treatment density of CD8+ T-cells, compared to baseline, is ≥6.7 on a log scale as determined by ROC curve analysis.

In still another embodiment, the present invention provides the biomarker for use as described in any of the previous embodiments, wherein the patient is likely to benefit in the form of PFS if the threshold value for the fold increase in CD8+ T-cells, compared to baseline, is ≥0.9 on a log scale and the threshold value for the on-treatment density of CD8+ T-cells, compared to baseline, is ≥6.2 on a log scale as determined by repeated landmark analysis.

In still another embodiment, the present invention provides the biomarker as defined in any of the embodiments above, for use in early stage clinical trials.

In still another embodiment, the present invention provides the biomarker as defined in any of the embodiments above for use in monitoring treatment of a patient, with cancer, wherein the treatment involves an immunotherapy.

In still another embodiment, the present invention provides the biomarker as defined in any of the embodiments above for use to enable the decision whether a patient, with cancer, continues treatment with immunotherapy.

In still another embodiment, the present invention provides a method of monitoring efficacy of an immunotherapy in a patient, with cancer, said method comprising measuring and calculating values for the FC and/or OTD of CD8 T-cells as defined in any of the embodiments above, comparing said values to the corresponding values at baseline, and continuing with said immunotherapy if said values are above the threshold values as also defined herein. In one embodiment, this method is an in vitro method.

In still another embodiment, the present invention provides a method of treating a cancer patient with an immunotherapy, said method comprising measuring and calculating values for the FC and/or OTD of CD8 T-cells as defined in any of the embodiments above, comparing said values to the corresponding values at baseline, and continuing with said immunotherapy if said values are above the threshold values as also defined herein.

In still another embodiment, the present invention provides an immunotherapy for use in treating a patient, with cancer, wherein the patient is selected for treatment, or for continuation of treatment with said immunotherapy, when the values for the FC and/or OTD of CD8 T-cells are above the threshold values as defined herein before when compared to their corresponding values at baseline,

The present invention will now be further illustrated by some working example for purposes of clarity of understanding. The descriptions and examples should not be construed as limiting the scope of the invention. The disclosures of all patent and scientific literature cited herein are expressly incorporated in their entirety by reference.

### EXAMPLES

### Materials and Methods

### Investigational Drugs Used

Simlukafusp alfa (FAP-IL2v), Emactuzumab (EMAC), Selicrelumab (CD40), Cergutuzumab Amunaleukin (CEA-IL2v) and an in-house investigational bispecific antibody directed to FAP-41BBL.

### Studies:

The analysis is based on samples and data collected from multicenter open-label phase I dose escalation studies and a phase II multicenter open-label basket trial (Study Identifiers:

NCT02627274 (FAP-IL2v), NCT03063762 (FAP-IL2v), NCT03386721 (FAP-IL2v), NCT02323191 (EMAC), NCT02665416 (CD40), NCT02304393 (CD40), NCT02350673 (CEA-IL2v), NCT04826003 (FAP-41BBL)).

The training cohort consisted entirely of matched biopsies from early-phase studies of Simlukafusp alfa (FAP-IL2v). Simlukafusp alfa (FAP-IL2v) is an immunocytokine comprising an antibody against fibroblast activation protein a (FAP) and an IL-2 variant with a retained affinity for IL-2Rβγ>IL-2 Rβγ and abolished binding to IL-2 Ra [8]. The validation set consisted of data and samples from early-phase studies.

### Assessments:

Tumor response was evaluated according to the RECIST 1.1 [9] criteria for solid tumors assessed every 6 weeks or 8 weeks for the first year and 12 weeks thereafter. The analysis of tumor biopsies were based on fresh tissue when available or archival tissue. The on-treatment schedule is dependent on the study. Details of the trials are provided in supplementary table 1.

### Immunohistochemical Staining and Digital Image Analysis

Staining of immune cell infiltrate and digital image analysis for quantification of cells immune cell infiltrate was performed as previously described [10]. Briefly, 2.5 μm thick sections were stained for single and double chromogenic assays for CD11b/CD14, CD11b/CD15, CD8/Ki67, ARG1, and FOXP3 using Ventana Discovery Ultra, Discovery XT, or Benchmark XT automated stainers (Ventana Medical Systems, Tucson, AZ) with NEXES version 10.6 software. Chromogenic reactions were performed with the appropriate, conjugated secondary antibodies and Discovery Purple, Discovery Yellow or OptiView DAB detection kit (Ventana MedicalSystems). Immunohistochemically stained slides were digitally scanned at 20× magnification with the high throughput iScan HT (Ventana Medical Systems). Whole-slide images were analyzed with the HALO Software (IndicaLabs) tool. Total cell counts, annotation areas and cell object XY coordinates were extracted for tumor, invasive margin and normal regions of interest (ROI).

### Statistical Analysis

The analyses included all patients treated with the investigational drugs, who had a biopsy at baseline and on-study. Values of total CD8 (baseline, on-treatment density (OTD) and fold change from baseline (FC)) were log-transformed in the analysis. ORR was analyzed using classification tables and receiver operating characteristic (ROC) curve analysis. The Youden's index was used for optimal threshold determination for the ORR endpoint [11]. Analyses of PFS were performed using a landmark cox PH hazard model (landmark time of 60 days, at which most of the on-treatment biopsies were taken) [12]. The C-index was used as a measure of association between the PFS and OTD or FC. For analyses of FC, the cox PH model included the baseline value and the OTD value. Impact of OTD and FC on tumor kinetic was performed using the Stein model with OT or FC as covariates. The data were separated into a training dataset (simlukafusp alfa studies) and a validation dataset (other studies). Threshold determination was performed on the training dataset and threshold performance was evaluated on the validation dataset.

### Example 1: Establishing and Validation of Threshold Values

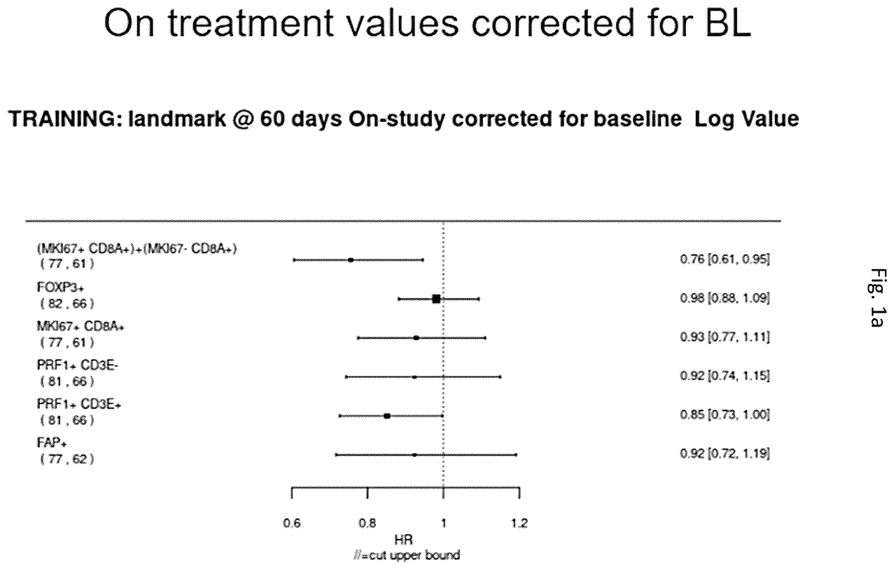

At first, evaluation in the training dataset (see below) took place as to whether CD8 on-treatment density with or without baseline correction has the strongest impact on risk of progression. The analysis identified that among 6 different biomarker in the tumor microenvironment, total CD8 density (MKI67+CD8A+)+(MKI67-CD8A+) followed by cytotoxic T cells (PRF1+ CD3+) density correlated best with the reduced risk of progression (see FIGS. 1A and 1B).

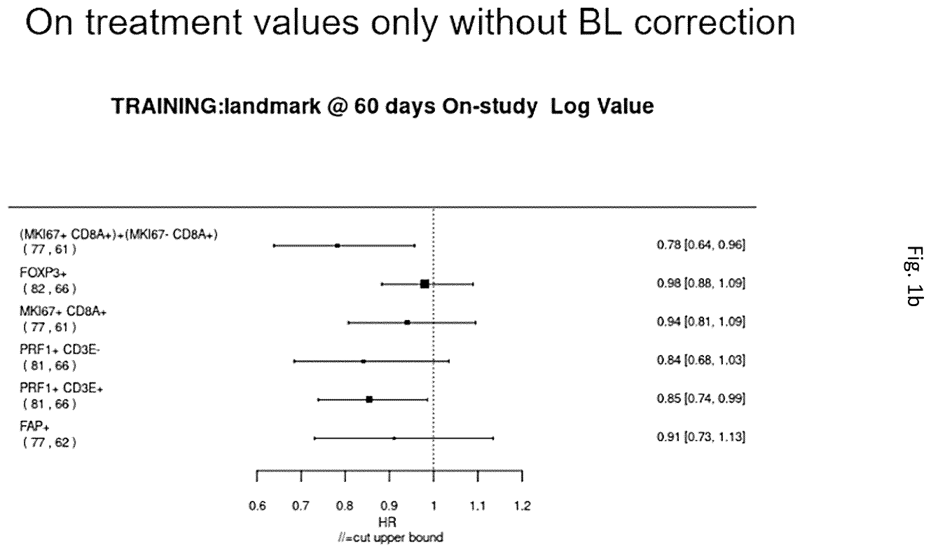

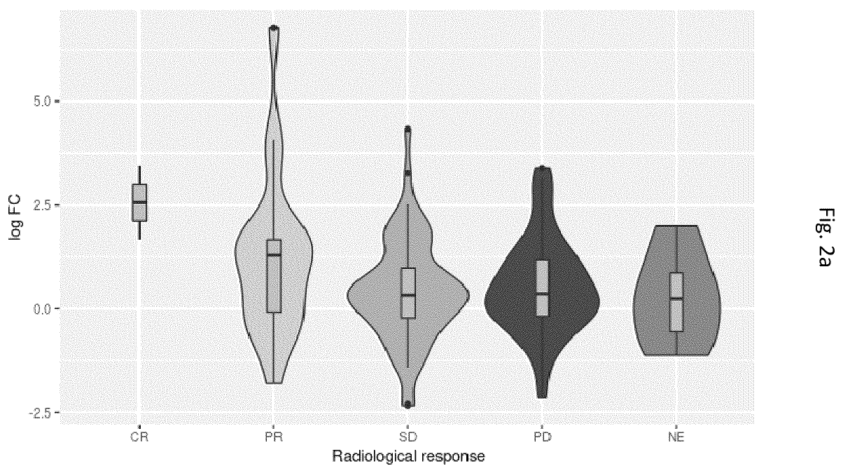

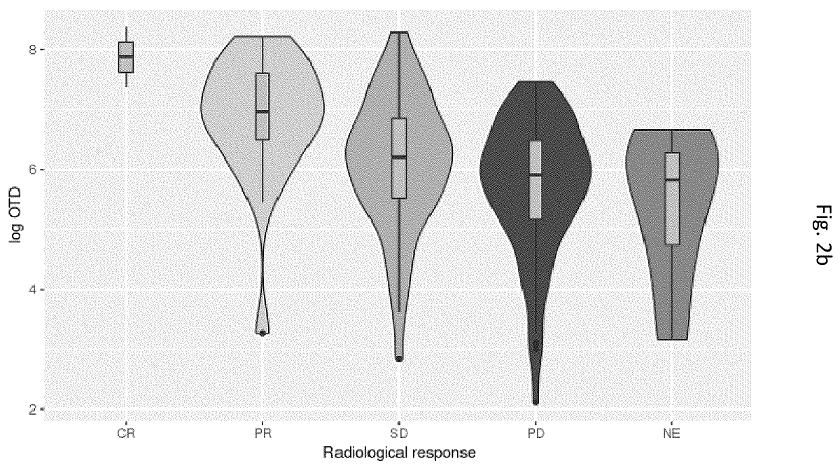

Then the association of both FC (fold change from baseline) and OTD (on-treatment density) of total CD8 T cells with clinical response was established. FIGS. 2A and 2B and Table 1 show the results of these analyses. For both FC and OTD, the value was higher for responding patients (complete response (CR), Partial response (PR)) in comparison to other response categories.

TABLE 1
Mean log FC and log OTD for each response category

| RESPf | mean(LPAIREDFCHG) | mean(LAVAL) | n() |
|---|---|---|---|
| <ord> | <dbl> | <dbl> | <int> |
| CR | 2.5589588 | 7.873589 | 2 |
| PR | 1.1966340 | 6.831429 | 21 |
| SD | 0.4293975 | 6.118365 | 59 |
| PD | 0.5174708 | 5.706500 | 66 |
| NE | 0.2470931 | 5.378311 | 7 |

For Progression Free Survival (PFS), an association was observed between FC and OTD given by the landmark analysis using the log of both FC and OTD as covariates. This association appears to be stronger for OTD than FC (see Table 2).

TABLE 2
Association between PFS and FC and OTD.

| Measurement | HR (95% CI) | Cindex |
|---|---|---|
| FC | 0.86 (0.73-1.025) | 0.53 |
| OTD | 0.75 (0.61-0.92) | 0.63 |

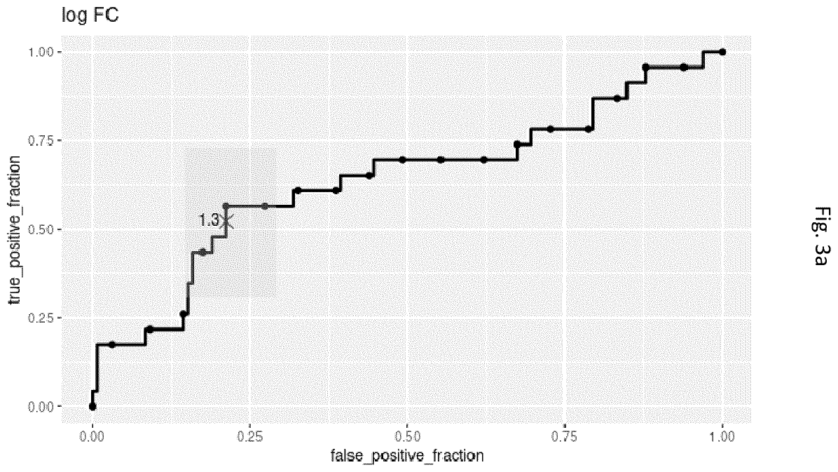

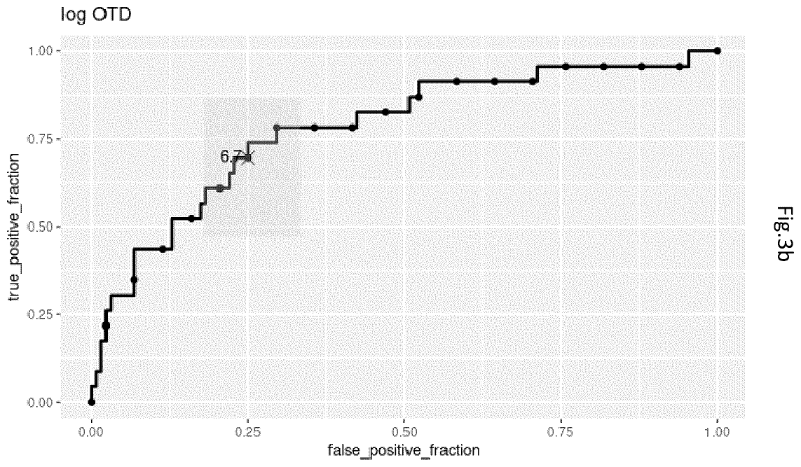

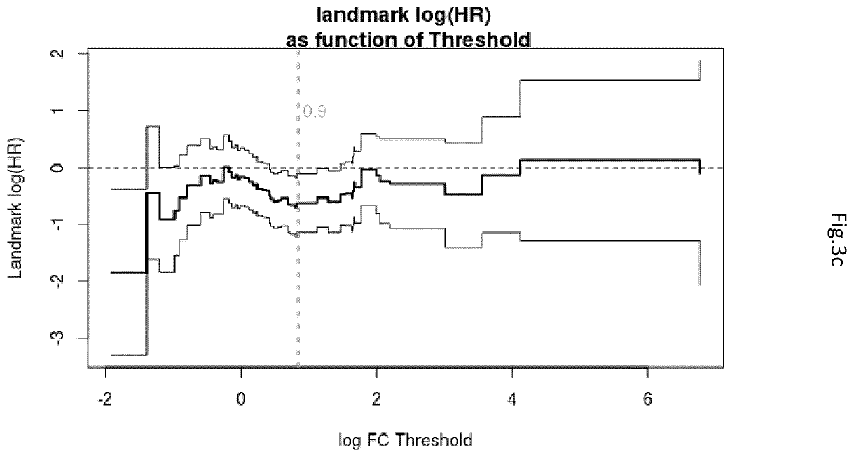

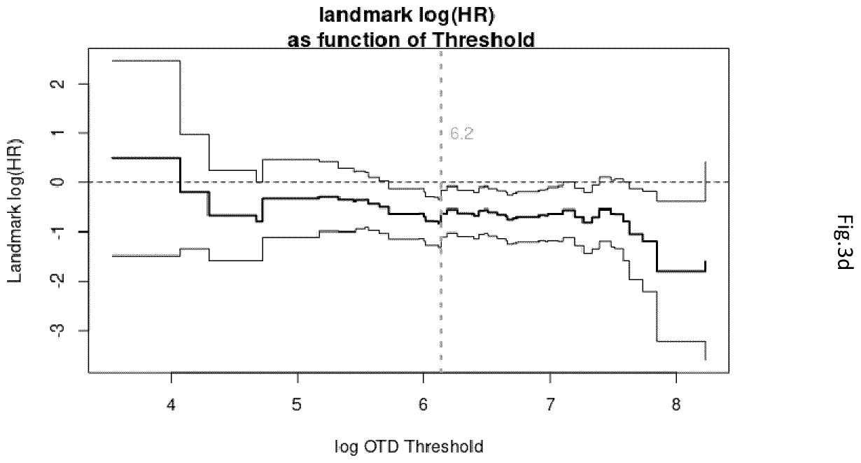

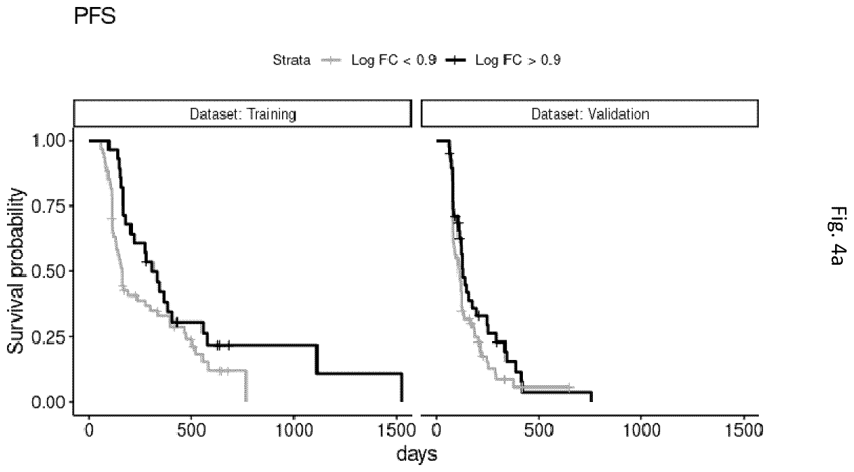

The threshold definition was achieved, on the training dataset, using a ROC curve analysis (on the rate of CR/PR) and repeated landmark analysis on PFS. The AUC under the ROC curve for FC was 0.63 with an optimal threshold of 1.3 log FC. For log OTD, AUC was 0.78 with the corresponding optimal threshold =6.7. For PFS, the lowest optimal threshold was obtained for log FC as 0.9 and log OTD =6.2 (see FIGS. 3A-3D). The thresholds derived from the rate of response (PR/CR) and PFS analysis were different, and the inventors chose to rely on the thresholds obtained from the PFS analysis. The rationale for this choice was that the PFS analysis is a more granular clinical endpoint and that the PFS is more representative of clinical efficacy in immunotherapies than the rate of PR/CR [4].

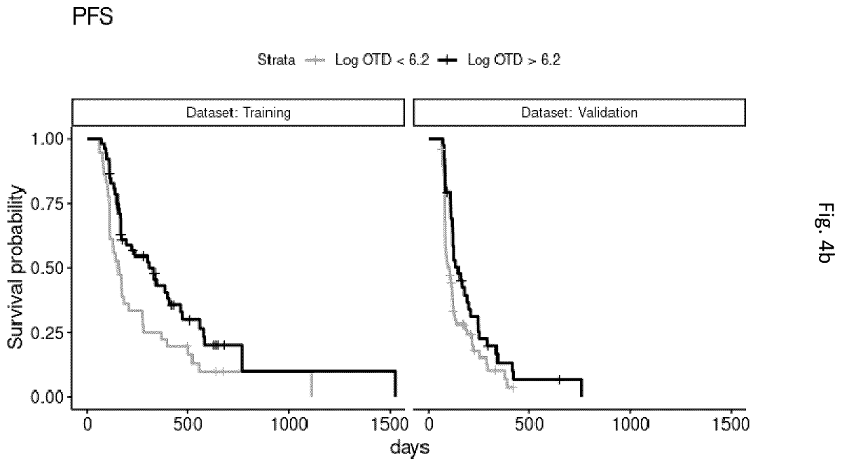

With these defined thresholds, FIGS. 4A and 4B and Table 3 show the differences between groups (below and above thresholds), in the training and validation datasets. PFS HR (95% CI) for

FC were 0.60 (0.36-1.0) for the training and 0.72 (0.46-1.10) for the validation dataset. For OTD, the values were 0.58 (0.36-0.93) for the training dataset and 0.64 (0.41-0.98) for the validation dataset. For both FC and OTD, Table 3 indicates that the non-responders are mostly classified in the below threshold groups (on the training and validation datasets) whereas, a majority of responders are classified in the above threshold group (on both training and validation datasets)

TABLE 3
Classification table for response on the
training (top) and validation (bottom)

| RidF IDM | Resp | No Resp | Total |
|---|---|---|---|
| Log FC < 0.9 | 10.0 (43.5%) | 94.0 (71.2%) | 104.0 (67.1%) |
| Log FC > 0.9 | 13.0 (56.5%) | 38.0 (28.8%) | 51.0 (32.9%) |
| Total | 23.0 (100.0%) | 132.0 (100.0%) | 155.0 (100.0%) |
| Log FC < 0.9 | 6.0 (46.2%) | 150.0 (72.1%) | 156.0 (70.6%) |
| Log FC > 0.9 | 7.0 (53.8%) | 58.0 (27.9%) | 65.0 (29.4%) |
| Total | 13.0 (100.0%) | 208.0 (100.0%) | 221.0 (100.0%) |
| Log OTD < 6.2 | 5.0 (21.7%) | 78.0 (59.1%) | 83.0 (53.5%) |
| Log OTD > 6.2 | 18.0 (78.3%) | 54.0 (40.9%) | 72.0 (46.5%) |
| Total | 23.0 (100.0%) | 132.0 (100.0%) | 155.0 (100.0%) |
| Log OTD < 6.2 | 4.0 (30.8%) | 165.0 (79.3%) | 169.0 (76.5%) |
| Log OTD > 6.2 | 9.0 (69.2%) | 43.0 (20.7%) | 52.0 (23.5%) |
| Total | 13.0 (100.0%) | 208.0 (100.0%) | 221.0 (100.0%) |

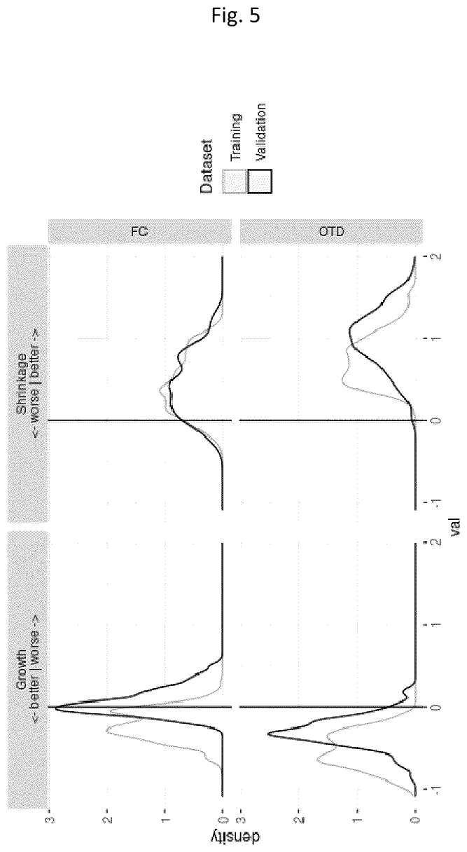

Tumor Growth kinetic is used to evaluate the validity of the threshold, in addition to PFS and response. FIG. 5 provides the distribution of the parameter capturing the effect of being above threshold. In this figure and for growth parameters, a distribution centered on the left indicates that patients above threshold would have a lower tumor growth than patients below threshold. For the shrinkage, a distribution centered on the right indicates that patients above threshold would have a larger tumor shrinkage. It can be seen that for FC, the effect of total CD8 on tumor growth and shrinkage is not marked, with no difference, for patients above threshold, in growth but increased shrinkage for the validation dataset and lower shrinkage for the training dataset. For the OTD endpoint, the effect of total CD8 is more clear, with clear smaller growth and higher shrinkage for patients above threshold.

Taken together, these results support the conclusion that a high value of FC and OTD were associated with improved clinical outcomes at a patient level.

### Example 2: Correlation of Decision Rules with Clinical Outcomes

In a next step, decision rules based on FC and/or OTD were investigated and the outcome of the decision rule correlated to the clinical outcome assessed by the study teams on each individual studies or cohorts (for the phase II study of FAP-IL2v). The decision rule could be based on the mean of the log FC or log OTD. In this case, the confidence level was defined as the posterior probability that the true log FC or log OTD was above the threshold given the observed data on the study. In the calculation of the posterior probability, the log FC or log OTD was assumed to be normal with variance given in Table 4. The variance was derived from the training and validation dataset pooled, although a sensitivity analysis shows that the variability was similar across datasets. A higher confidence level provided a higher chance of improved clinical outcome (as established by the patient level association described above). A second option was to define the decision rule based on the observed number of patients that are above threshold. Then the confidence level was defined as the posterior probability that the number of patients above threshold was above the target rate. This target rate is set a priori as the expected number of patients with sufficient increase in total CD8 T cells and was defined based on preclinical experiments.

TABLE 4
Standard deviation observed on our dataset
and used in the confidence level definition

| Measurement | Sd |
|---|---|
| log FC | 1.4 |
| log OTD | 1.44 |

Decision rules were derived from these confidence level as follows: Positive outcome with confidence level above 75%, indecisive outcome with confidence level between 50% and 75% and negative outcome with confidence levels below 50%. These levels are standard in Bayesian decision making in early clinical trials (see e.g. Fisch et al. [15]). Both decision rules were evaluated in Table 5 for FC at the study level. In this Table, the studies utilized for the training and validation datasets are ordered according to the confidence level. The color shade indicates a confidence level >75% (light-grey), between 50-75% (grey) and <50% (dark-gey). The frames indicate cohorts in which a clinical decision could be made (sufficient sample size of 10-100 patients). The light-grey frames highlight the cohorts for which clinical efficacy was positive. The grey frames highlight the cohorts for which the efficacy was borderline (in-between), i.e., not sufficient for further development but higher than standard of care (SOC). The dark-grey frames indicate the drugs that have insufficient clinical efficacy (see also legend in Table 5). 2 decision rules were compared: the rule based on the geometric mean of FC to clinical efficacy and the rule based on number of patients above this threshold, which requires specification of the target rate. The geometric mean of log FC to clinical efficacy led to a more consistent outcome with the selected cohorts all ranking high for light-grey followed by grey and then dark-grey frames.

For these reasons, we chose to focus further evaluation of the decision rule using the rule based on the geometric mean of FC.

The decision rule could be applied to mean log OTD and is given in Table 6. Despite a better association between total CD8 and clinical efficacy at the patient level compared to log FC, the decision rule appears to be weaker among the cohorts, with inconsistent decisions with respect to clinical efficacy.

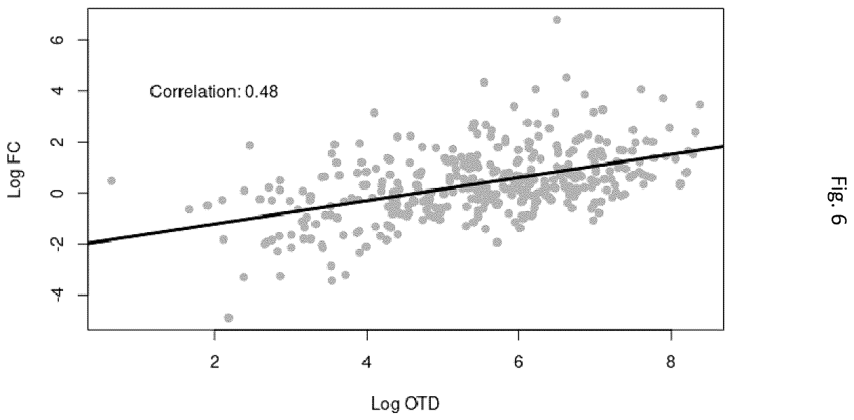

FIG. 6 provides the correlation between log OTD and log FC. Acknowledging the fact that log FC include log OTD in its definition, a moderate correlation was observed between the log FC and log OTD indicating that both endpoints may be relevant for deriving more robust decision rule. Combining both log OTD and log FC endpoints could be done in multiple ways. It was chosen to combine the OTD and log FC decision rules rather than using a score with the log baseline value. The benefit is that the decision rule would leverage the FC as such rather than through the OTD.

The drawback is that it relies on the correlation between the distributions of log FC and log OTD, which can be estimated from the used dataset.

From the joint distribution of log FC and log OTD, a combined decision rule can be reached using two methods: It can be considered either that log FC and log OTD need to be above threshold to obtain a positive outcome, or that either one of log FC or log OTD is sufficient to obtain a positive outcome. Both approaches are illustrated on our datasets in Table 7.

Table 7 demonstrates that the requirement of having both log FC and log OTD above threshold is too stringent, and results in low confidence level obtained by this combined decision rule. In contrast, a decision rule whereby either log FC or log OTD being above threshold is associated with better clinical outcome, improves the confidence interval. The lower panel of table 7 shows that a decision rule based on either log FC or log OTD being above threshold is much more consistent with the clinical assessment than a rule requiring both log FC and log OTD above threshold.

The data thus suggest a combined decision rule based on either log FC or log OTD endpoints.

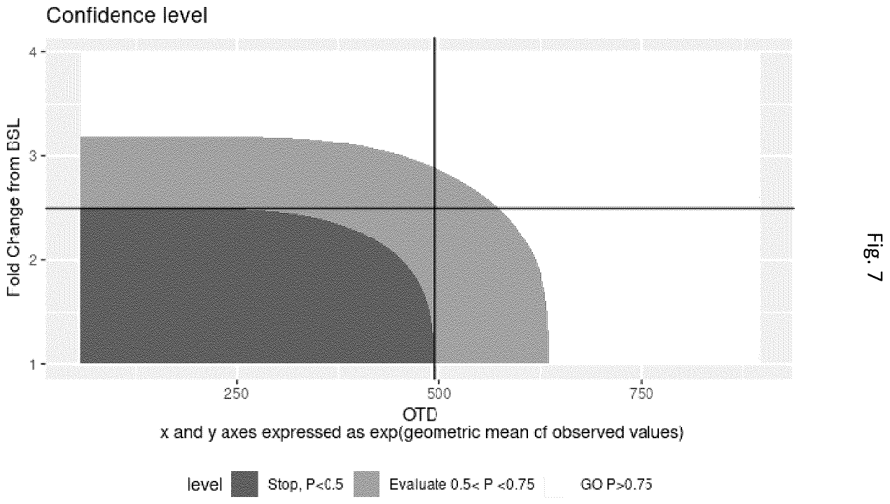

Given this decision rule, and assuming a sample size of 15 patients with paired biopsies, the readout of one experiment is given in FIG. 7. This figure shows the decision that would be reached from the observed data in the experiment.

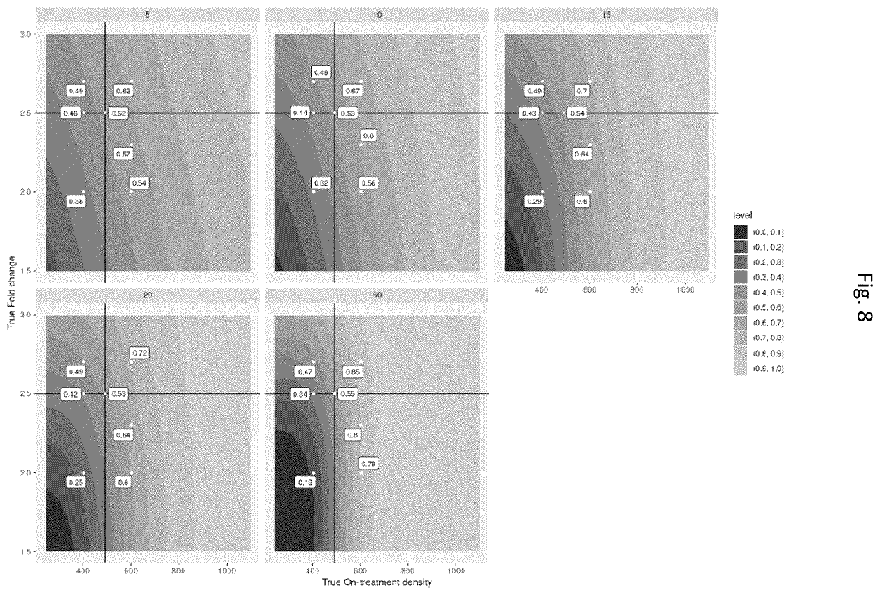

One can also compute the probability of success (i.e. a confidence level >0.75) for a given true log OTD and log FC. This calculation accounts for the fact that even the true values are never observed in reality, but only random experiments are observed. FIG. 8 display the probability of success for studies with 5, 10, 20, 60 patients with paired biopsies. This figure shows the increase in probability of success when the true values are above the threshold (upper right quadrant) and decrease when the true values are below (lower left quadrant).

### REFERENCES

1. Rosenberg S A. IL-2: the first effective immunotherapy for human cancer. Journal of immunology, 2014; 192(12), 5451-8.
2. Huang P W, Chang J W. Immune checkpoint inhibitors win the 2018 Nobel Prize. Biomedical journal, 2019; 42(5), 299-306.
3. Ascierto P A, McArthur G A. Checkpoint inhibitors in melanoma and early phase development in solid tumors: what's the future? Journal of translational medicine 2017; 15(1), 173.
4. Anagnostou V, Yarchoan M, Hansen A R, Wang H, Verde F, Sharon E, et al. Immuno-oncology Trial Endpoints: Capturing Clinically Meaningful Activity. Clinical Cancer Research, 2017; 23(17), 4959-69.
5. Galon J, Fridman W H, Pages F. The adaptive immunologic microenvironment in colorectal cancer: a novel perspective. Cancer Research, 2007; 67(5), 1883-6.
6. Galon J, Pagès F, Marincola F M, Angell H K, Thurin M, Lugli A, et al. Cancer classification using the Immunoscore: a worldwide task force. Journal of translational medicine, 2012; 10, 205.
7. Petrelli F, Ghidini M, Costanzo A, Rampulla V, Varricchio A, Tomasello G. Surrogate endpoints in immunotherapy trials for solid tumors. Ann Transl Med, 2019; 7(7), 154.
8. Waldhauer I, Gonzalez-Nicolini V, Freimoser-Grundschober A, Nayak T K, Fahrni L, Hosse R J, et al. Simlukafusp alfa (FAP-IL2v) immunocytokine is a versatile combination partner for cancer immunotherapy, mAbs, 2021;13(1), 1913791-1 to 1913791-13.
9. Eisenhauer E A, Therasse P, Bogaerts J, Schwartz L H, Sargent D, Ford R, et al. New response evaluation criteria in solid tumours: revised RECIST guideline (version 1.1). European journal of cancer, 2009; Vol. 45(2), 228-47.
10. Zwing N, Failmezger H, Ooi C-H, Hibar D P, Cañamero M, Gomes B, et al. Analysis of Spatial Organization of Suppressive Myeloid Cells and Effector T Cells in Colorectal Cancer-A Potential Tool for Discovering Prognostic Biomarkers in Clinical Research, Frontiers in immunology, 2020; 11, doi:10.3389/fimmu.2020.550250.
11. Fluss R, Faraggi D, Reiser B. Estimation of the Youden Index and its associated cutoff point. Biometrical journal (Biometrische Zeitschrift), 2005; 47(4), 458-472.
12. Houwelingen H C V. Dynamic Prediction by Landmarking in Event History Analysis, Scandinavian Journal of Statistics, 2007; 34(1), 70-85.
13. Harrell F E, Lee K L, Mark D B JSim. Multivariable prognostic models: issues in developing models, evaluating assumptions and adequacy, and measuring and reducing errors, Statistics in Medicine, 1996; Vol. 15, No. 4, 361-387.
14. Stein W D, Gulley J L, Schlom J, Madan R A, Dahut W, Figg W D, et al. Tumor regression and growth rates determined in five intramural NCI prostate cancer trials: the growth rate constant as an indicator of therapeutic efficacy, Clinical Cancer Research, 2011; 17(4), 907-917.
15. Fisch R, Jones I, Jones J, Kerman J, Rosenkranz G K, Schmidli H. Bayesian Design of Proof-of-Concept Trials, Therapeutic innovation & regulatory science, 2015; Vol. 49, No. (1), 155-162.
16. Tumeh P C, Harview C L, Yearley J H, Shintaku I P, Taylor E J M, Robert L, et al. PD-1 blockade induces responses by inhibiting adaptive immune resistance, Nature 2014; 515(7528), 568-571.
17. Bocchialini G, Schiefer A I, Müllauer L, Thanner J, Bauer J, Thaler F, et al. Tumour immune microenvironment in resected thymic carcinomas as a predictor of clinical outcome, British Journal of Cancer, 2022, 127(6), 1162-1171. doi: 10.1038/s41416-022-01875-7
18. Shimizu S, Hiratsuka H, Koike K, Tsuchihashi K, Sonoda T, Ogi K, et al. Tumor-infiltrating CD8(+) T-cell density is an independent prognostic marker for oral squamous cell carcinoma, Cancer medicine, 2019; 8(1), 80-93.
19. Fang J, Li X, Ma D, Liu X, Chen Y, Wang Y, et al. Prognostic significance of tumor infiltrating immune cells in oral squamous cell carcinoma. BMC cancer, 2017; 17(1), 375.
20. Galon et al., Science, 2006, 313 (5795), 1960-1964.
21. Swets, J. A. and Pickett, R. M.; Evaluation of Diagnostic Systems: Methods from Signal Detection Theory; 1982; New York: Academic Press.
22. Anderson J. R., Cain K. C., Gelber, R. D.; Analysis of survival by tumor response; J. Clin. Oncol. 1, 1983, 710-719.

1. A method to determine whether a patient, with cancer, is likely to benefit from treatment with immunotherapy comprising
a) obtaining samples from that patient before (at baseline) and after treatment,
b) determining the fold increase (FC) in CD8+ T-cells and/or the on-treatment density (OTD) of CD8+ T-cells in the sample after treatment compared to the sample at baseline,
c) comparing values obtained in b) to threshold values, wherein
the patient is likely to benefit from said treatment if the values obtained in b) are above the threshold.

2. The method of claim 1, which is an in vitro method.

3. The method of claim 1 or 2, wherein step b) comprises determining the fold increase in CD8+ T-cells or the on-treatment density of CD8+ T-cells in the sample after treatment compared to the sample at baseline

4. The method of any one of claims 1 to 3, wherein the sample is a patient's tumor biopsy sample.

5. The method of any one of claims 1 to 4, wherein the cancer is a solid tumor.

6. The method of claim 5 wherein the solid tumor is selected from lung cancer (including non-small cell lung cancer), breast cancer, thyroid cancer, head and neck cancer, pancreatic cancer, prostate cancer, bladder cancer, colon cancer and colorectal cancer.

7. The method of any one of claims 1 to 6, wherein the patient benefit from treatment is either of a partial response (PR), a complete response (CR) or a longer progression-free survival (PFS).

8. The method of any one of claims 1 to 7, wherein the patient is likely to benefit in form of PFS if the threshold value for the fold increase in CD8+ T-cells, compared to baseline, is ≥0.9 on a log scale as determined by repeated landmark analysis.

9. The method of any one of claims 1 to 7, wherein the patient is likely to benefit in the form of PR, CR and/or PFS if the threshold value for the on-treatment density of CD8+ T-cells, compared to baseline, is ≥6.2 on a log scale (approximately 500 cells/mm²) as determined by repeated landmark analysis.

10. The method of any one of claims 1 to 7, wherein the patient is likely to benefit from immunotherapy if
the threshold value for the fold increase in CD8+ T-cells, compared to baseline, is ≥0.9 on a log scale as determined by ROC curve analysis, and
the threshold value for the on-treatment density of CD8+ T-cells, compared to baseline, is ≥6.2 on a log scale (approximately 500 cells/mm²) as determined by repeated landmark analysis.

11. The method of any one of claims 1 to 7, wherein the patient is likely to benefit from a PR or CR if the threshold value for the fold increase in CD8+ T-cells, compared to baseline, is ≥1.3 on a log scale and the threshold value for the on-treatment density of CD8+ T-cells, compared to baseline, is ≥6.7 on a log scale as determined by ROC curve analysis.

12. The method of any one of claims 1 to 7, wherein the patient is likely to benefit in the form of PFS if the threshold value for the fold increase in CD8+ T-cells, compared to baseline, is ≥0.9 on a log scale and the threshold value for the on-treatment density of CD8+ T-cells, compared to baseline, is ≥6.2 on a log scale as determined by repeated landmark analysis.

13. The method of any one of claims 1 to 12 for use in early stage clinical trials.

14. The method of any one of claims 1 to 12 for use in monitoring treatment of a patient, with cancer, wherein the treatment involves an immunotherapy.

15. The method of any one of claims 1 to 12 for use to enable the decision whether a patient, with cancer, continues treatment with immunotherapy.

16. A biomarker for use in a method according to any one of claims 1 to 15, wherein the biomarker is characterized by
the fold increase (FC) in CD8+ T-cells; and/or
the on-treatment density (OTD) of CD8+ T-cells
in a sample obtained from that patient after treatment, compared to a sample of that same patient at baseline.
